(19) United States
(12) Patent Application Publication
(10) Pub. No.: US 2026/0176409 A1
(43) Pub. Date: Jun. 25, 2026

(54) Process for the Depolymerization of Polyethylene Terephthalate (PET)

(71) Applicant: MANGALORE REFINERY & PETROCHEMICALS LTD., Mangalore (IN)

(72) Inventors: Sanjay Pandurang KAMBLE, Pune (IN); Karthick RAMALINGAM, Mangalore (IN); Nishant Nandkishore MARKANDEYA, Pune (IN); Sunil Sitaram BHONGALE, Pune (IN); Pramod Kumar HEGDE, Mangalore (IN); Manjunatha Megur Ganesh BHAT, Mangalore (IN); Sathyanarayana Hanakere CHANNAIAH, Mangalore (IN)

(21) Appl. No.: 19/539,148

(22) Filed: Feb. 13, 2026

Related U.S. Application Data
(63) Continuation of application No. PCT/IB2024/056593, filed on Jul. 6, 2024.

(30) Foreign Application Priority Data
Aug. 19, 2023 (IN) 202341055663

(51) Int. Cl. C08G 18/42; C08G 18/75; C08G 63/695; C08J 11/24

(52) U.S. Cl. CPC C08G 18/4252; C08G 18/755; C08G 63/6956; C08J 11/24; C08J 2367/02

(57) ABSTRACT

The present disclosure relates to a process for the depolymerization of polyethylene terephthalate (PET). The process of the present disclosure is performed without using any catalyst or ionic liquids. Further, the reagents used in the process can be recovered and reused.

### CROSS REFERENCE TO RELATED APPLICATIONS

This application is a Bypass Continuation of PCT Patent Application No. PCT/IB2024/056593 having International filing date of Jul. 6, 2024, which claims the benefit of priority of Indian Patent Application number 202341055663, filed Aug. 19, 2023, the contents of which are all incorporated herein by reference in their entirety.

### FIELD

The present disclosure relates to depolymerization. Particularly, the present disclosure relates to a process for the depolymerization of polyethylene terephthalate (PET).

### BACKGROUND

The background information herein below relates to the present disclosure but is not necessarily prior art.

A variety of plastic/polymeric materials are used in packaging products, electronic and electrical equipments and household products. Most of the plastics are also consumed in the construction and automotive industries. There are two main types of plastics, one of which are thermoplastics, which softens after heating and hardens after cooling, and thermosets, which hardens when cured and cannot be re-moulded again. Thermoplastic polymer mainly offers ideal properties such as its transparency, cleanliness, food safety, toughness, barrier properties and competitive cost which makes them suitable for various applications such as packaging and insulation.

However, plastic materials and their by-products pollute cities and oceans and cause human and animal health problems. Researchers estimated that billions of tons of plastic are produced for decades. Most of the plastics end up in landfills and accumulated in the surroundings. If the current trend continues, it is also projected that plastics will require a major part of the total global oil.

Conventionally, almost 80% of the plastics used are thermoplastics. Polyethylene terephthalate (PET) has become the world's most used packaging material for food and beverages. In order to overcome pollution, PET recycling is a major task. Conventionally, almost 90% of PET is recycled mechanically; however, it has limitation that it can't be recycled for multiple cycles. Other than mechanical recycling, chemical recycling methods are currently used such as alcoholysis which generally involves the use of supercritical methanol and ethanol which ultimately leads to the production of DMT (dimethyl terephthalate) and DET (diethyl terephthalate) respectively. Other than alcoholysis, aminolysis, ammonolysis, hydrolysis and glycolysis are also used in chemical recycling. Aminolysis generally involves the use of excess ethanolamine and leads to the production of BHETA (bis(2-hydroxyethyl terephthalamide). Ammonolysis involves the use of ammonia leading to the formation of terephthalohydrazide (TPHD). Hydrolysis involves the use of water in an acidic, alkaline or neutral medium which generally yields TPA (Terephthalic acid), a monomer for PET production along with ethylene glycol.

Furthermore, glycolysis has gained much attention as the process generally yields bis(2-hydroxyethyl) terephthalate (BHET) which is a main monomer for PET production. The bis (2-hydroxyethyl) terephthalate (BHET) monomer is generally produced by using the esterification process of TPA (terephthalic acid) and EG (ethylene glycol) or transesterification process of DMT (dimethyl terephthalate) and EG. However, the conventional glycolysis processes are associated with drawbacks such as mandatory use of ionic liquids and catalysts, and performance of the reaction at higher temperature and pressure conditions.

There is, therefore, felt a need to provide a process for depolymerization of PET that mitigates the drawbacks mentioned hereinabove or provide at least a useful alternative.

### OBJECTS

Some of the objects of the present disclosure, which at least one embodiment herein satisfies, are as follows:

An object of the present disclosure is to ameliorate one or more problems of the background or to at least provide a useful alternative.

Another object of the present disclosure is to provide a process for the depolymerization of polyethylene terephthalate (PET).

Still object of the present disclosure is to provide a process for the depolymerization of PET which requires less reaction time.

Yet another object of the present disclosure is to provide a process for the depolymerization of PET without the use of ionic liquids.

Still another object of the present disclosure is to provide a process for the depolymerization of PET which does not require catalyst.

Yet another object of the present disclosure is to provide a process for the depolymerization of PET, wherein the solvent and co-solvent used in the process can be recycled and reused in multiple cycles.

Still another object of the present disclosure is to provide a process for the depolymerization of PET which can be performed at lower temperature and pressure conditions.

Yet another object of the present disclosure is to provide a simple, sustainable, cost effective, eco-friendly process for the depolymerization of waste PET.

Still another object of the present disclosure is to provide a process for the depolymerization of PET which provides a pure BHET monomer.

Other objects and advantages of the present disclosure will be more apparent from the following description, which is not intended to limit the scope of the present disclosure.

### SUMMARY

The present disclosure relates to a process for the depolymerization of PET (polyethylene terephthalate). In the process, polyethylene terephthalate (PET) flakes are mixed with a solvent and a co-solvent in a predetermined weight ratio to obtain a first mixture. The first mixture is then heated in an inert atmosphere, at a temperature in the range of 150° C. to 210° C. and at a pressure in the range of 35 mbar to 6 bar under stirring at a first predetermined speed for a first predetermined time period to obtain a second mixture comprising a crude of bis(2-hydroxyethyl) terephthalate (BHET) monomers and oligomers, and a solvent mixture. Thereafter, the solvent mixture is separated from the crude to obtain a separated solvent mixture and a separated crude. At least one fluid medium is added to the separated crude in a predetermined ratio and stirred at a second predetermined speed for a second predetermined time period to solubilize the BHET monomers to obtain floccules of the oligomers. The floccules of the oligomers are then separated to obtain a cake and a third mixture comprising BHET monomers and the fluid medium. The BHET monomers from the third mixture are isolated.

In an embodiment of the present disclosure, the process is a continuous process or a batch process.

In an embodiment, the solvent is ethylene glycol.

In an embodiment, the co-solvent is at least one selected from the group consisting of tetrahydrofuran, formamide, N-methylformamide, N,N-dimethylformamide, dimethylacetamide (DMAc), dimethylcarbonate (DMC), dimethyl sulfoxide (DMSO), 1-Methylimidazole and N-Methyl-2-pyrrolidone (NMP).

In an embodiment, the co-solvent is at least one selected from the group consisting of dimethylformamide (DMF), dimethylacetamide (DMAc) and dimethylcarbonate (DMC).

In an embodiment, the predetermined weight ratio of polyethylene terephthalate (PET) flakes to the solvent to the co-solvent is in the range of 1:5:5 and 1:10:10.

In an embodiment of the present disclosure, the predetermined weight ratio of polyethylene terephthalate (PET) flakes to the solvent to the co-solvent is 1:7.5:7.5.

In an embodiment, the first predetermined speed is in the range of 500 rpm to 1500 rpm.

In an embodiment, the first predetermined speed is 1000 rpm.

In an embodiment, the first predetermined time period is in the range of 20 minutes to 120 minutes.

In an embodiment, the first predetermined time period is 30 minutes.

In an embodiment, the solvent mixture is separated from the crude by distillation at a temperature in the range of 50° C. to 240° C., and at a pressure in the range of 10 mbar to 1020 mbar.

In an embodiment, the solvent mixture is separated from the crude by distillation at a temperature in the range of 70° C. to 200° C., and at a pressure of 800 mbar.

In an embodiment, the fluid medium is at least one selected from the group consisting of water, methanol, ethanol and acetonitrile.

In an embodiment, the fluid medium is water.

In an embodiment, a ratio of the fluid medium to the crude is in the range of 5:1 to 25:1.

In an embodiment, the ratio of the fluid medium to the crude is 11:1.

In an embodiment, the second predetermined agitation speed is in the range of 100 rpm to 700 rpm.

In an embodiment, the second predetermined time period is in the range of 5 minutes to 30 minutes.

In an embodiment, the second predetermined agitation speed is 500 rpm.

In an embodiment, the second predetermined time period is 15 minutes.

In an embodiment, the BHET monomers are isolated by using at least one method selected from crystallization, drying, evaporation and flash distillation.

In an embodiment, the crystallization is performed at a temperature in the range of 0° C. to 10° C.; the drying by distillation is performed at a temperature in the range of 70° C. to 100° C. at a pressure in the range of 100 mbar to 1020 mbar; and the evaporation or the flash distillation is performed at a temperature in the range of 50° C. to 110° C. and at a pressure in the range of 35 mbar to 1020 mbar.

In an embodiment, the obtained BHET monomer is characterized by having a purity greater than 98%, a crystallinity greater than 67% and a particle size less than 10 microns.

In an embodiment, the obtained BHET monomer is characterized by having a purity of 98.75%, a crystallinity in the range of 67.5% to 72.2% and a particle size in the range of 0.2 microns to 7.3 microns.

### BRIEF DESCRIPTION OF ACCOMPANYING DRAWINGS

FIG. 1 illustrates the calibration curve for BHET monomers prepared in accordance with an embodiment of the present disclosure by using a high-performance liquid chromatography (HPLC);

FIG. 2 illustrates pictorial representation of (A) PET flakes used for the reaction, (B) Oligomers obtained after water addition, and (C) BHET (monomer) crystals obtained after crystallization in accordance with an embodiment of the present disclosure;

FIG. 3 illustrates a high-performance liquid chromatography (HPLC) chromatogram of a crude in accordance with an embodiment of the present disclosure;

FIG. 4 illustrates a high-performance liquid chromatography (HPLC) chromatogram of pure BHET in accordance with an embodiment of the present disclosure;

FIG. 5 illustrates a graphical representation of % selectivity vs time (minutes) for co-solvent screening in accordance with an embodiment of the present disclosure;

FIG. 6 illustrates a graphical representation of % PET conversion and % BHET selectivity vs reaction time (minutes) in accordance with an embodiment of the present disclosure;

FIG. 7 illustrates a graphical representation of % PET conversion and % BHET selectivity vs reaction temperature (° C.) in accordance with an embodiment of the present disclosure;

FIG. 8 illustrates a graphical representation of N₂ pressure vs % PET conversion and % BHET selectivity in accordance with an embodiment of the present disclosure;

FIG. 9 illustrates a graphical representation of ethylene glycol (EG) weight ratio vs % PET conversion and % BHET selectivity in accordance with an embodiment of the present disclosure;

FIG. 10 illustrates a graphical representation of dimethylformamide (DMF) weight ratio vs % PET conversion and % BHET selectivity in accordance with an embodiment of the present disclosure;

FIG. 11 illustrates a graphical representation of speed of agitation vs % PET conversion and % BHET in accordance with an embodiment of the present disclosure;

FIG. 12 illustrates FTIR spectra of BHET in accordance with an embodiment of the present disclosure;

FIG. 13 illustrates 1H NMR spectra of BHET in accordance with an embodiment of the present disclosure;

FIG. 14 illustrates 13C NMR spectra of BHET in accordance with an embodiment of the present disclosure;

FIG. 15 illustrates DSC thermograms of PET, isolated BHET and isolated oligomer in accordance with an embodiment of the present disclosure; and

FIG. 16 illustrates FESEM micrographs of (A) Untreated (fresh) PET (B) PET treated with EG, (C) PET treated with DMF in accordance with an embodiment of the present disclosure.

### DETAILED DESCRIPTION

The present disclosure relates depolymerization. Particularly, the present disclosure relates to a process for the depolymerization of polyethylene terephthalate (PET).

Embodiments of the present disclosure will now be described with reference to the accompanying drawings.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Conventionally, almost 80% of the plastics used are thermoplastics. Polyethylene terephthalate (PET) has become the world's most used packaging material for food and beverages. In order to overcome pollution, PET recycling is a major task. Conventionally, almost 90% of PET is recycled mechanically. Other than mechanical recycling, chemical recycling methods are currently used such as alcoholysis which generally involves the use of supercritical methanol and ethanol which ultimately leads to the production of DMT (Dimethyl Terephthalate) and DET (Diethyl Terephthalate) respectively. Other than alcoholysis, aminolysis, ammonolysis, hydrolysis and glycolysis are also used in chemical recycling. Aminolysis generally involves the use of excess ethanolamine and leads to the production of BHETA (bis(2-hydroxyethyl terephthalamide). Another process ammonolysis involves the use of ammonia leading to the formation of terephthalohydrazide (TPHD). Hydrolysis involves the use of water in an acidic, alkaline or neutral medium which generally yields TPA (Terephthalic acid), a monomer for PET production along with ethylene glycol.

Furthermore, glycolysis has gained much attention as the process generally yields bis(2-hydroxyethyl) terephthalate (BHET) which is a main monomer for PET production. The bis (2-hydroxyethyl) terephthalate (BHET) monomer is generally produced by using the esterification process of TPA (terephthalic acid) and EG (ethylene glycol) or transesterification process of DMT (dimethyl terephthalate) and EG. However, the conventional glycolysis processes are associated with drawbacks such as mandatory use of ionic liquids and catalysts, and performance of the reaction at higher temperature and pressure conditions.

The present disclosure provides a process for the depolymerization of polyethylene terephthalate (PET).

In the process of the depolymerization of polyethylene terephthalate (PET, polyethylene terephthalate (PET) flakes are mixed with a solvent and a co-solvent in a predetermined weight ratio to obtain a first mixture. The first mixture is then heated in an inert atmosphere, at a temperature in the range of 150° C. to 210° C. and at a pressure in the range of 35 mbar to 6 bar under stirring at a first predetermined speed for a first predetermined time period to obtain a second mixture comprising a crude of bis(2-hydroxyethyl) terephthalate (BHET) monomers and oligomers, and a solvent mixture. Thereafter, the solvent mixture is separated from the crude to obtain a separated solvent mixture and a separated crude. At least one fluid medium is added to the separated crude in a predetermined ratio and stirred at a second predetermined speed for a second predetermined time period to solubilize the BHET monomers to obtain floccules of the oligomers. The oligomer floccules are then separated to obtain a cake and a third mixture comprising BHET monomers and the fluid medium. The BHET monomers from the third mixture are isolated.

In an embodiment of the present disclosure, the process is a continuous process or a batch process.

The process is described in detail herein below:

In a first step, polyethylene terephthalate (PET) flakes are mixed with a solvent and a co-solvent in a predetermined weight ratio to obtain a first mixture.

In accordance with an embodiment of the present disclosure, the predetermined weight ratio of the polyethylene terephthalate (PET) flakes to ethylene glycol (EG) to the co-solvent is in the range of 1:5:5 to 1:10:10. In an exemplary embodiment of the present disclosure, the weight ratio (PET:EG:DMF) is 1:7.5:7.5.

In accordance with an embodiment of the present disclosure, the solvent is ethylene glycol.

In accordance with the present disclosure, the co-solvent is at least one selected from the group consisting of acetamide, furan, formamide, imidazole and pyrrolidone.

In an embodiment of the present disclosure, the co-solvent is at least one selected from the group consisting of tetrahydrofuran, formamide, N-methylformamide, dimethylformamide (DMF), dimethylacetamide (DMAc), dimethylcarbonate (DMC), dimethyl sulfoxide (DMSO), 1-methylimidazole and N-methyl-2-pyrrolidone (NMP). In the exemplary embodiment of the present disclosure, the co-solvent is dimethylformamide (DMF). In another exemplary embodiment of the present disclosure, the co-solvent is dimethylacetamide (DMAc). In still another exemplary embodiment of the present disclosure, the co-solvent is dimethylcarbonate (DMC).

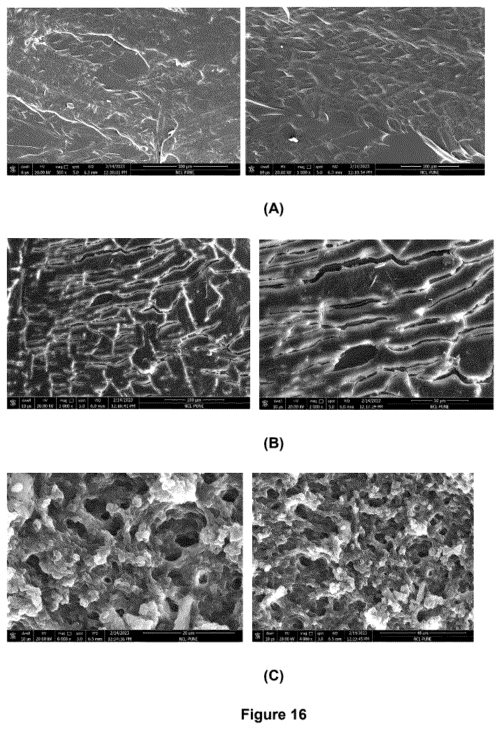

In the process of the present disclosure, the inventors have surprisingly found that the solvent ethylene glycol (EG) helps in achieving the desired selectivity, and the co-solvent helps to achieve the desired conversion in less reaction time, when the weight ratio of the polyethylene terephthalate (PET) flakes to ethylene glycol (EG) to the co-solvent is maintained in the range of 1:5:5 to 1:10:10. Hence, PET depolymerization is achieved without the use of ionic liquids and catalysts. In accordance with an exemplary embodiment of the present disclosure, the FESEM micrograph of PET treated with DMF of FIG. 16 clearly shows that DMF assists in penetration of EG into PET flakes thereby increasing the rate of depolymerization.

In an exemplary embodiment, polyethylene terephthalate (PET) flakes are mixed with a solvent and a co-solvent mixed in an autoclave (high pressure) reactor.

In a second step, the first mixture is heated in an inert atmosphere, at a temperature in the range of 150° C. to 210° C. and at a pressure in the range of 35 mbar to 6 bar under stirring at a first predetermined speed for a first predetermined time period to obtain a second mixture comprising a crude of bis(2-hydroxyethyl) terephthalate (BHET) monomers and oligomers, and a solvent mixture.

In an exemplary embodiment, the temperature is 185° C. and the pressure is 5 bar.

In an exemplary embodiment, the inert atmosphere is nitrogen atmosphere.

In accordance with an embodiment of the present disclosure, the first predetermined speed is in the range of 500 rpm to 1500 rpm. In the exemplary embodiment, the first predetermined speed is 1000 rpm.

In accordance with an embodiment of the present disclosure, the first predetermined time period is in the range of 20 minutes to 120 minutes. In the exemplary embodiment, the first predetermined time period is 30 minutes.

The depolymerization of polyethylene terephthalate to bis(2-hydroxyethyl) terephthalate (BHET) monomers is provided below:

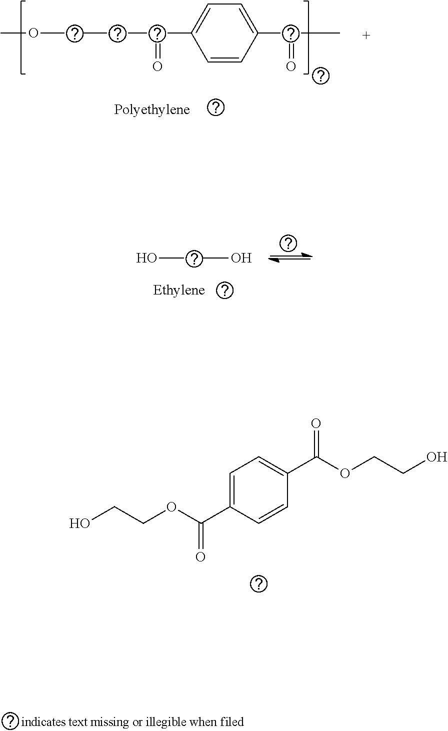

In a third step, the solvent mixture from the crude is separated to obtain a separated solvent mixture and a separated crude.

In accordance with an embodiment of the present disclosure, the solvent mixture is separated from the crude (comprising BHET monomers and oligomers) by distillation at a temperature in the range of 50° C. to 240° C. and at a pressure in the range of in the range of 10 mbar to 1020 mbar. In the exemplary embodiment of the present disclosure, when DMF is used as a co-solvent, the distillation temperature is 153° C. and the pressure is 800 mbar. In another exemplary embodiment of the present disclosure, when DMC is used as a co-solvent, the distillation temperature is 90° C. and the pressure is 800 mbar. In still another exemplary embodiment of the present disclosure, when DMAc is used as a co-solvent, the distillation temperature is 165° C. and the pressure is 800 mbar.

In an embodiment of the present disclosure, the solvent mixture is continuously recycled and mixed with PET flakes for obtaining the first mixture, thereby making the process efficient, economic, and environment friendly.

In a fourth step, at least one fluid medium is added to the separated crude in a predetermined ratio and stirring at a second predetermined speed for a second predetermined time period to solubilize the BHET monomers and floccules of the oligomers are obtained.

In accordance with an embodiment of the present disclosure, the addition of fluid medium completely recovers the floccules of oligomers.

In accordance with an embodiment of the present disclosure, the fluid medium is a solubilizing solvent for selectively solubilizing the BHET monomers.

In accordance with an embodiment of the present disclosure, the fluid medium is at least one selected from the group consisting of water, methanol, ethanol and acetonitrile. In an exemplary embodiment of the present disclosure, the fluid medium is water.

In accordance with an embodiment of the present disclosure, the ratio of the fluid medium to the crude is in the range of 5:1 to 25:1. In an exemplary embodiment of the present disclosure, the ratio of the fluid medium to the crude is 11:1.

In accordance with an embodiment of the present disclosure, the second predetermined stirring speed is in the range of 100 rpm to 700 rpm. In an exemplary embodiment, the second predetermined stirring speed is 500 rpm.

In accordance with an embodiment of the present disclosure, the second predetermined time period is in the range of 5 minutes to 30 minutes. In an exemplary embodiment of the present disclosure, the second predetermined time period is 15 minutes.

In a fifth step, the oligomer floccules are separated to obtain a cake and a third mixture comprising BHET monomers and the fluid medium.

In an embodiment of the present disclosure, the oligomer floccules are separated by filtration or gravity settling to obtain separated oligomer floccules and the third mixture comprising BHET monomers and the fluid medium. In an exemplary embodiment of the present disclosure, the oligomer floccules are separated by filtration.

In a sixth step, the BHET monomers from the third mixture are isolated.

In accordance with an embodiment of the present disclosure, the isolation of BHET monomers is done by at least one method selected from crystallization, drying, evaporation and flash distillation. In an exemplary embodiment of the present disclosure, the isolation of BHET monomers from the fluid medium is done by crystallization.

In accordance with an embodiment of the present disclosure, the crystallization is performed at a temperature in the range of 0° C. to 10° C. In an exemplary embodiment, the crystallization temperature is 5° C.

In accordance with another embodiment of the present disclosure, the drying by distillation is performed at a temperature in the range of 70° C. to 100° C. and at a pressure in the range of 100 mbar to 1020 mbar. The drying by distillation is performed at a vacuum and an atmospheric pressure as well.

In accordance with an embodiment of the present disclosure, the evaporation is performed at a temperature in the range of 50° C. to 110° C. and at a pressure in the range of 35 mbar to 1020 mbar.

In accordance with an embodiment of the present disclosure, the flash distillation is performed at a temperature in the range of 50° C. to 110° C. and at a pressure in the range of 35 mbar to 1020 mbar.

In accordance with an embodiment of the present disclosure, the solvent mixture comprising solvent and co-solvent is recycled and reused for depolymerization of PET in multiple cycles.

In accordance with an embodiment of the present disclosure, the fluid medium after the separation of BHET monomers is recycled and reused multiple times to solubilize BHET monomers from the separated crude in the second mixture.

The process of the present disclosure provides a BHET product having comparatively high purity. In accordance with the present disclosure, the BHET product is characterized by having a purity greater than 98%.

In accordance with an exemplary embodiment of the present disclosure, the purity of BHET monomer crystals obtained is 98.75%.

Further, upon crystallization, the BHET product has a crystallinity greater than 67% and a particle size less than 10 microns.

The % crystallinity of BHET is determined by the following mathematical formula:

$$‘ \% crystallinity = \frac{Area of crystalline peaks}{Total area ( of crystalline and amorphous region )} ⋆ 100$$

In an exemplary embodiment, the purity of the BHET product is 98.75% pure, has a crystallinity in the range of 67.5% to 72.2%, and particle size in the range of 0.2 microns to 7.3 microns.

In an exemplary embodiment of the present disclosure, the process is a continuous process. The process comprises a step of mixing polyethylene terephthalate (PET) flakes, ethylene glycol (solvent) and dimethylformamide (DMF) (co-solvent) in a 1:7.5:7.5 (a predetermined weight ratio) to obtain a first mixture. The first mixture is heated in an inert (N₂) atmosphere, at 185° C., at 5 bar and at 1000 rpm (a first predetermined speed) for 30 minutes (a first predetermined time period) to obtain a second mixture comprising a crude of bis(2-hydroxyethyl) terephthalate (BHET) monomers and oligomers, and a solvent mixture. The solvent mixture from the crude is separated by using rotary evaporator at 153° C., at 800 mbar to obtain a separated solvent mixture and separated crude. In this step, excess ethylene glycol and DMF are recovered to obtain a separated crude. After the solvent recovery, water is added to the separated hot crude in a ratio of 11:1 (water: separated crude) and is continuously stirred at 500 rpm (a second predetermined agitation speed) for 15 minutes (a second predetermined time period) to solubilize the BHET monomers to obtain floccules of the oligomers. The floccules of the oligomers are separated to obtain a cake and a third mixture comprising BHET monomers and water (the fluid medium). The third mixture is then subjected to crystallization at 5° C. to obtain BHET crystals (BHET isolation).

The process of the present disclosure for the depolymerization of PET is performed in a comparatively lesser reaction time. Furthermore, the conventional processes mandatorily require ionic liquids and catalyst to achieve the desired depolymerization in the desired time period. However, the process of the present disclosure is performed without the use of ionic liquids or catalysts and at lower temperature and pressure along with the separation and purification of monomers.

The foregoing description of the embodiments has been provided for purposes of illustration and is not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results obtained can be extrapolated to industrial scale.

### EXPERIMENTAL DETAILS

### Experiment 1: Process for the Depolymerization of Polyethylene Terephthalate (PET) to Obtain Monomers (BHET)

200 g of polyethylene terephthalate flakes were mixed with 1500 g of ethylene glycol (solvent) and 1500 g of dimethylformamide (DMF) (co-solvent) to obtain a first mixture and fed in an autoclave (high pressure) reactor. The first mixture was then heated in an inert (N₂) atmosphere with 5 bar pressure at 185° C. at an agitation speed of 1000 rpm (a first predetermined agitation speed) for 30 minutes (first predetermined time) in the autoclave to obtain a second mixture comprising a crude of bis(2-hydroxyethyl) terephthalate (BHET) monomers and oligomers and a solvent mixture.

3200 g of the so obtained second mixture comprising the crude and the solvent mixture was fed to rotary evaporator at 153° C. at a pressure of 800 mbar for solvent recovery step. In this step, excess ethylene glycol and DMF were recovered (2884 g) to obtain a separated crude. After the solvent recovery, the separated hot crude (288 g) was fed to a vessel which was continuously stirred at speed 500 rpm (second predetermined agitation speed) for 15 minutes (second predetermined time period) and 3264 g of water (fluid medium) was added to solubilize the monomer to obtain floccules of the oligomers. The oligomers in the form of floccules were recovered completely with carried over BHET monomers.

The so obtained oligomer floccules were then filtered to obtain a cake (floccules of the oligomers) and a third mixture comprising BHET monomers and water (fluid medium). The wet cake (floccules of the oligomers) of 324 g on drying resulted in 90 g of dry oligomers and contained BHET monomers.

3174 g of the third mixture containing BHET monomers and water (filtrate) was then subjected to crystallization at 5° C. to obtain BHET crystals. The crystallization process yielded wet BHET mass of 252 g, which on drying yielded 150 g of dry BHET. Further, mother liquor (2904 g) after separating the crystals, was concentrated for the recovery of the remaining BHET and after second crystallization, around 12 g of BHET was recovered from the mother liquor.

After each reaction, the PET conversion was determined by using the following formula:

$$\% Conversion = \frac{Initial PET weight - Final PET weight}{Initial PET weight} \times 100$$

Isolated yield of BHET was calculated by the following equation:

$$\% Isolated Yield = \frac{Amount of BHET Isolated}{Amount of BHET in reaction crude} \times 100$$

$$\% Isolated Yield = \frac{219}{266} \times 100$$

$$\% Isolated Yield = 82.3 \%$$

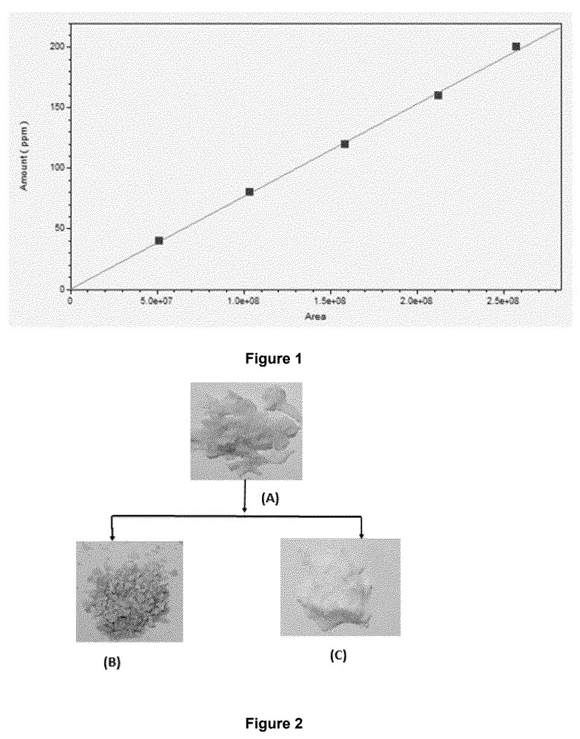

The third mixture (i.e. crude of BHET monomers and oligomers) was analyzed by using High Performance Liquid Chromatography (HPLC) at a flow rate of 1 ml/minute, by using 70:30 Methanol:water (v/v) solvent system and at a wavelength of 240 nm (maximum absorbance for BHET). HPLC calibration was performed by using standard BHET by diluting the standard samples in the methanol-water mixture of 70:30 v/v and varying the concentration from 40 mg/L to 200 mg/L. The BHET calibration chart is shown in FIG. 1.

The pictorial representation of (A) PET flakes used for the reaction, (B) oligomers obtained after water addition and (C) BHET monomer crystals obtained after crystallization is depicted in FIG. 2.

Further, the so obtained oligomers were characterized by using differential scanning calorimetry (DSC) and the BHET monomers were characterized by Differential scanning calorimetry (DSC), Nuclear magnetic resonance (NMR) and Fourier transform infrared (FTIR).

The so obtained BHET monomers had a crystallinity in the range of 67.5% to 72.2% and a particle size in the range of 0.2 microns to 7.3 microns.

### HPLC Chromatogram of Crude Comprising of BHET Monomers and Oligomers:

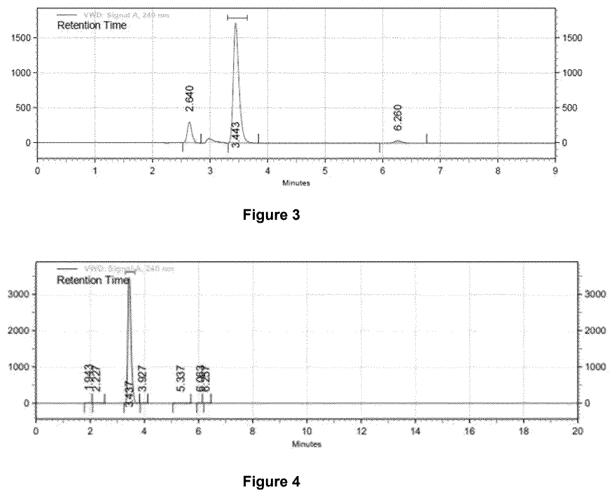

The second mixture comprising the crude of BHET monomers, and oligomers; and solvent mixture as obtained in experiment 1 was further subjected to HPLC analysis. FIG. 3 illustrates the result of the analysis.

The results of FIG. 3 showed the peak at a retention time (RT) of 3.4 minutes which corresponds to the BHET (monomer), the peak at an RT of 6.2 minutes which corresponds to dimers, a small peak at 2.9 minutes that is attributed to DMF solvent and the peak at 2.6 minutes which corresponds to impurity. From these peaks it is evident that the PET was depolymerized to obtain BHET monomers, dimers (oligomers), DMF solvent and other impurities

### HPLC Chromatogram of Pure BHET:

The pure BHET in accordance with the present disclosure was further subjected to HPLC analysis. FIG. 4 illustrates the result of the analysis.

The results of FIG. 4 showed that BHET obtained had a purity of 98.75% and a significant reduction in area was observed for oligomers, DMF and impurities. Therefore, the purification of BHET is an important step to achieve the purity of 98.75% of BHET.

### Experiment 2: Study for Optimization of Co-Solvents and Solvent to Co-Solvent Ratios, Effect of N₂ Pressure, Effect of Speed of Agitation, Effect of Time, and Effect of Temperature

Various parameters were tested for studying the effect on the depolymerization of PET.

### 1. Optimization of Co-Solvents

The experiment 1 was performed using various co-solvents as listed in Table 1 and the reaction was performed at a temperature of 185° C., under inert atmosphere pressure (N₂ pressure 5 bar) at a speed of agitation 1000 rpm for various time period from 30 minutes to 2 hours. Table 1 illustrates the % selectivity and BHET weight % for different solvents. Further, the solvent mixture was separated to recover solvents.

TABLE 1
% selectivity and BHET weight % for different solvents:

| | DMAc | | DMF | | DMC | |
|---|---|---|---|---|---|---|
| | Dimethyl Acetamide | | Dimethyl Formamide | | Dimethyl carbonate | |
| Time | % BHET | BHET | % BHET | BHET | % BHET | BHET |
| (min) | selectivity | wt % | selectivity | wt % | Selectivity | wt % |
| 30 | 71.35 | 2.16 | 85.96 | 7.66 | 33.4 | 0.14 |
| 60 | 82.46 | 6.66 | 81.78 | 6.91 | 46.67 | 2.65 |
| 120 | 81.88 | 7.72 | 72.86 | 6.63 | 15.36 | 1.44 |

From Table 1, it is observed that the highest % selectivity and weight % of BHET product were 85.96% and 7.66%, respectively in the presence of DMF as a solvent in 30 minutes of reaction time. The same is evident from FIG. 5.

### 2. Optimization of Time for the PET Depolymerization

The experiment 1 was performed at various time periods to see the effect of time on depolymerization of PET. The reaction was carried out at a temperature of 185° C. under inert conditions (N₂ pressure 5 bar), at speed of agitation 1000 rpm and PET-EG-DMF weight ratio of 1:7.5:7.5. Table 2 shows the effect of time on the depolymerization of PET.

TABLE 2
Effect of time on depolymerization of PET

| Time | % PET | % BHET | Wt % |
|---|---|---|---|
| (min) | Conversion | Selectivity | BHET |
| 20 | 78 | 75.06 | 5.31 |
| 30 | 92 | 85.96 | 7.66 |
| 60 | 100 | 82.19 | 6.91 |
| 120 | 100 | 72.43 | 6.63 |

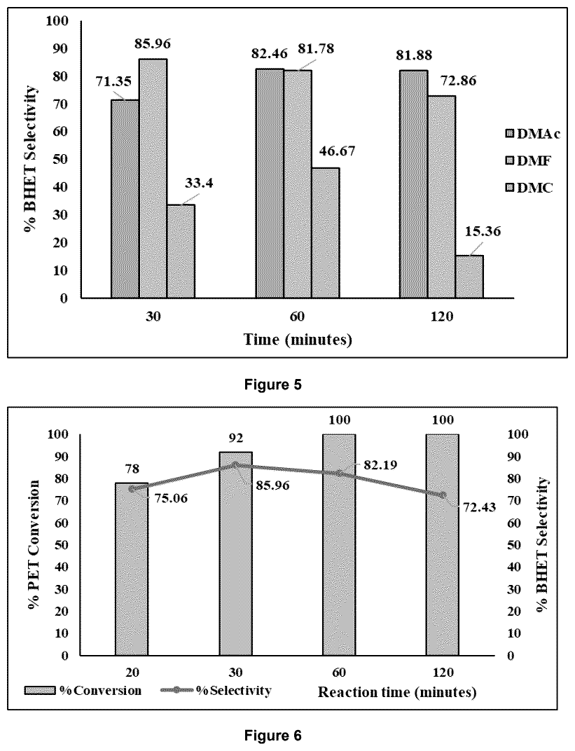

From Table 2, it is observed that with increase in reaction time, the selectivity as well as the weight % of the desired product BHET monomers was decreasing because the formation of impurities increases with increase in reaction time. Therefore, 30 minutes of depolymerization time gave the maximum % selectivity of 85.96 and BHET weight % of 7.66. The same is evident from FIG. 6.

### 3. Optimization of Temperature

The experiment 1 was carried out to at various temperatures to observe the effect of reaction temperature for 30 minutes of reaction time at 5 bar N₂ Pressure, speed of agitation of 1000 rpm and PET-EG-DMF ratios of 1:7.5:7.5 (by weight). The reaction temperature was varied from 170° C. to 200° C. Table 3 shows the effect of temperature on the depolymerization of PET in view of % PET Conversion and % BHET selectivity.

TABLE 3
Effect of temperature on the depolymerization of PET
in view of % PET Conversion and % BHET Selectivity

| Temperature | % PET | % BHET | Wt. % |
|---|---|---|---|
| (° C.) | Conversion | Selectivity | BHET |
| 170 | 68 | 83.56 | 5.18 |
| 180 | 92 | 85.96 | 7.66 |
| 185 | 100 | 89.7 | 8.55 |
| 190 | 100 | 83.24 | 7.51 |
| 200 | 100 | 76.62 | 6.675 |

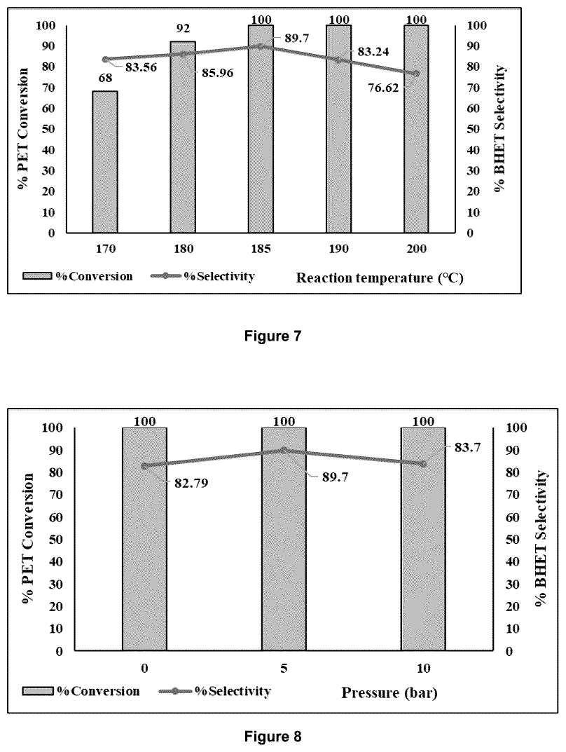

From Table 3, it is observed that at reaction temperature 185° C., maximum selectivity of BHET of 89.7% and weight % of BHET of 8.55 were achieved. The same is evident from FIG. 7.

### 4. Optimization of Nitrogen Pressure:

The experiment 1 was carried out to at various nitrogen pressures to check the effect of nitrogen. The reaction was carried out at temperature 185° C. for 30 minutes with PET to solvent to co-solvent weight ratios of 1:7.5:7.5 (PET-EG-DMF) and at speed of agitation 1000 RPM. Table 4 shows the effect of pressure on % PET Conversion and % BHET selectivity.

TABLE 4
Effect of Pressure on % PET Conversion and % BHET Selectivity

| Pressure | | | Wt % |
|---|---|---|---|
| (bar) | % Conversion | % Selectivity | of BHET |
| 0 | 100 | 82.79 | 7.46 |
| 5 | 100 | 89.7 | 8.55 |
| 10 | 100 | 83.7 | 7.64 |

From Table 4, it is observed that in the absence of nitrogen, the formation of impurities was more, which further led to a decrease in % BHET selectivity and in the presence of nitrogen (5 bar N₂ pressure) maximum selectivity observed is 89.7%. The same is evident from FIG. 8.

### 5. Optimization of PET to Solvent Ratio (PET to EG Weight Ratios)

The experiment 1 was carried at various ratios of PET to solvent ratio (ethylene glycol) and by keeping the amount of co-solvent (DMF) constant. The reaction was carried out at a temperature of 185° C., under an inert atmosphere (N₂ pressure 5 bar) for 30 min and at a speed of agitation 1000 RPM. Table 5 shows the effect of EG weight ratios on % PET conversion and % BHET selectivity.

TABLE 5
Effect of solvent (EG) weight ratios on
% PET conversion and % BHET selectivity

| Weight Ratio | | | |
|---|---|---|---|
| PET:Solvent:Co-solvent | % PET | % BHET | Wt % |
| (PET:EG:DMF) | Conversion | Selectivity | BHET |
| 1:5:7.5 | 100 | 75.6 | 7.36 |
| 1:6:7.5 | 100 | 75.03 | 7.4 |
| 1:7.5:7.5 | 100 | 89.7 | 8.56 |
| 1:9:7.5 | 100 | 83.82 | 6.94 |
| 1:10:7.5 | 100 | 81.34 | 6.61 |

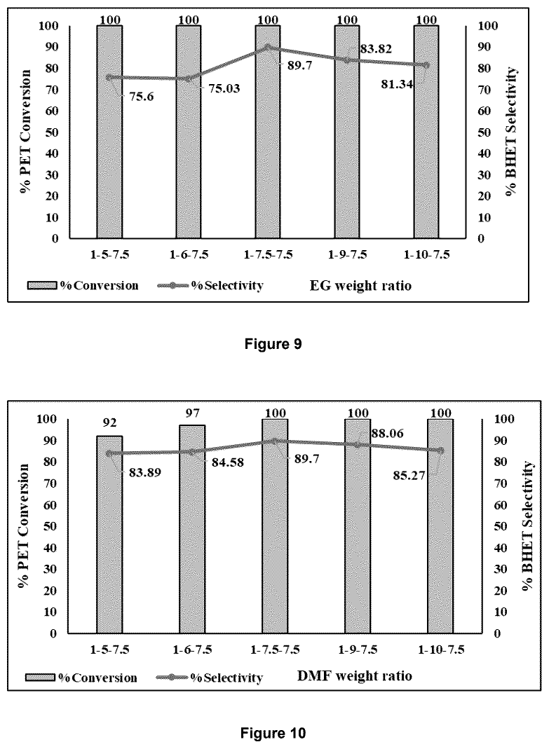

From Table 5, it is observed that the maximum 89.7% BHET selectivity was obtained at 1:7.5:7.5 weight ratio and with further increase in EG weight ratios, % BHET selectivity decreases, which may be due to the formation of side products/impurities. The same is evident from FIG. 9.

### 6. Optimization of PET to Co-Solvent Ratio (PET to DMF Weight Ratios)

The experiment 1 was carried at various ratios of PET to co-solvent ratio (DMF) and by keeping the amount of solvent (ethylene glycol) constant. The reaction was carried out at a reaction temperature of 185° C. under an inert atmosphere (N₂ pressure 5 bar) for 30 min and at a speed of agitation 1000 RPM. Table 6 shows the effect of DMF weight ratios on % PET conversion and % BHET selectivity.

TABLE 6
Effect of co-solvent (DMF) weight ratios on
% PET conversion and % BHET Selectivity

| Weight Ratio | % PET | % BHET | Wt % |
|---|---|---|---|
| (PET:EG:DMF) | Conversion | Selectivity | BHET |
| 1:7.5:5 | 92 | 83.89 | 8.56 |
| 1:7.5:6 | 97 | 84.58 | 8.27 |
| 1:7.5:7.5 | 100 | 89.7 | 8.56 |
| 1:7.5:9 | 100 | 88.06 | 7.47 |
| 1:7.5:10 | 100 | 85.27 | 6.86 |

From Table 6, it is observed that the maximum 89.7% BHET selectivity was obtained at 1:7.5:7.5 weight ratios. Further, with an increase in DMF weight ratio, % BHET selectivity decreases which may be due to the formation of unwanted side products (impurities). Also, at lower weight ratios of DMF, incomplete conversion was observed which may be due to the less dissolution of PET. The same is evident from FIG. 10.

### 7. Optimization of Speed of Agitation

The experiment 1 was carried at various agitation speed to observe the effect of the speed of agitation. The reaction was carried out at 185° C. under an inert atmosphere (N₂ pressure 5 bar) for 30 min and at a weight ratio of 1:7.5:7.5. Table 7 shows the effect of speed of agitation on % PET conversion and % BHET selectivity.

TABLE 7
Effect of speed of agitation on % PET
conversion and % BHET selectivity

| Speed of | | | |
|---|---|---|---|
| agitation | % PET | % BHET | Wt % |
| (RPM) | Conversion | Selectivity | BHET |
| 500 | 100 | 79.5 | 7.15 |
| 1000 | 100 | 89.7 | 8.56 |
| 1400 | 100 | 88.19 | 8.2 |

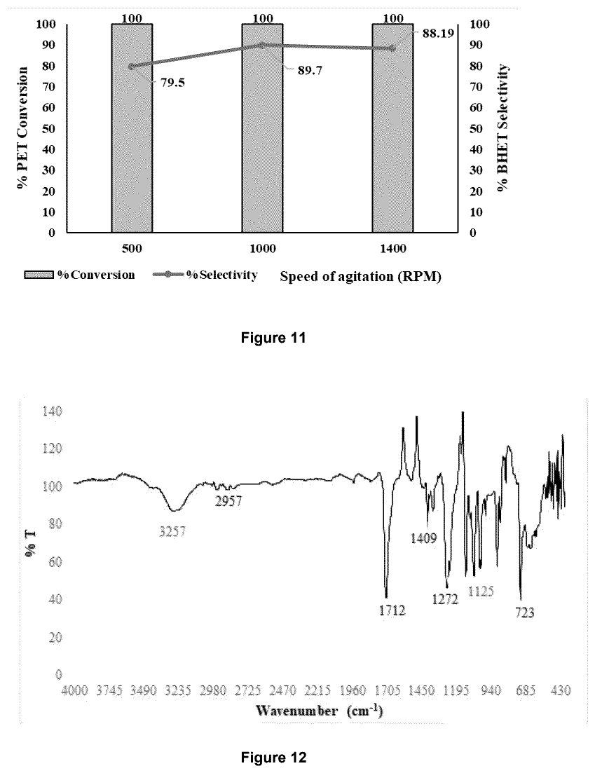

From Table 7, it is observed that the maximum 89.7% BHET selectivity was obtained at 1000 rpm and with further increase in speed there is no increase in BHET selectivity and wt % BHET formation. The same is evident from FIG. 11.

### Experiment 3: Characterization of Products

### FTIR Spectra of BHET

FIG. 12 illustrates the FTIR spectra of BHET which confirmed the functional groups present in the compound wherein the strong peak at 3257 cm⁻¹ represents O—H band, the peak at 2957 cm⁻¹ represents the vibration of alkyl C—H bonds, the peak at 1712 cm⁻¹ denotes C═O stretching vibration and the peak at 1272 cm⁻¹ denotes C—O vibration. Also, the peak at 1125 cm⁻¹ denotes C—O bond oxy association and the peaks in the range of 723 cm⁻¹ denote the vibration of the benzene ring.

### ¹H NMR Spectra of BHET

TABLE 8
Details of 1H NMR spectra of BHET

| Chemical Shift (ppm) | Interpretation |
|---|---|
| Single peak at 8.18 δ | Four aromatic benzene ring protons |
| Triplet at 5.12 δ | Two hydroxyl group protons |
| Triplet at 4.38 δ | Methylene groups protons connected to |
| | carbonyl groups |
| Quartet at 3.78 δ | Methylene groups protons connected to |
| | hydroxyl groups |

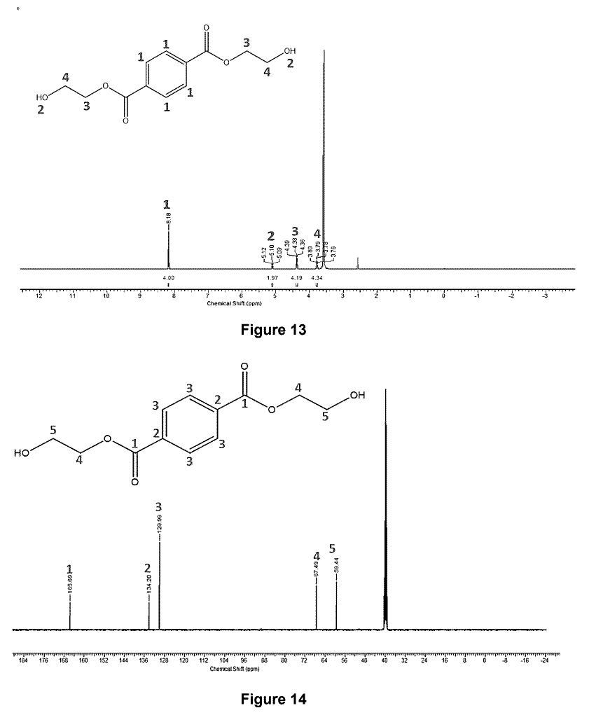

From Table 8 and FIG. 13, it is observed that the chemical shift data having single peak at 8.18 δ, triplet at 5.12 δ, triplet at 4.38 δ and quarter at 3.78 δ is for four aromatic benzene ring protons, two hydroxyl group protons, methylene groups protons connected to carbonyl groups, and methylene groups protons connected to hydroxyl groups, respectively, indicating that the product formed is BHET.

### ¹³C NMR Spectra of BHET

TABLE 9
Details of ¹³C NMR Spectra of BHET

| Chemical | | |
|---|---|---|
| Shift (ppm) | | Interpretation |
| 165.69 | δ | Carboxyl carbon |
| 134.2 | δ | Quaternary carbon attached to carboxylate group |
| 129.99 | δ | aromatic carbon atoms |
| 67.49 | δ | Two methylene carbons connected to carboxyl group |
| 59.44 | δ | Two methylene carbons connected to hydroxyl group |

From Table 9 and FIG. 14, it is observed that the chemical shifts at 165.69 δ, 134.2 δ, 129.99 δ, 67.49 δ and 59.44 δ are indicative of the presence of carboxyl carbon, quaternary carbon attached to carboxylate group, aromatic carbon atoms, two methylene carbons connected to carboxyl group and two methylene carbons connected to hydroxyl group, respectively, which clearly shows the presence of BHET.

### DSC Thermograms

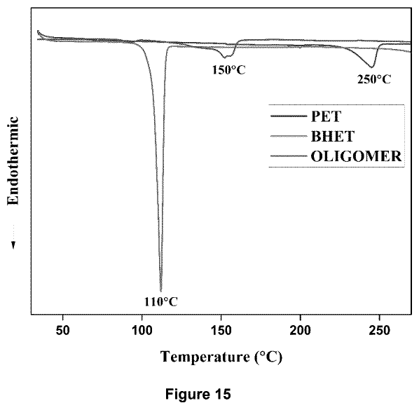

FIG. 15 showed Differential scanning calorimetry (DSC) thermograms of PET, isolated oligomer and isolated BHET. It is confirmed from FIG. 15 that the conversion of PET was 100%. It is also observed that some amount of BHET was lost along with oligomer and from the DSC thermogram of isolated BHET. Further it is confirmed that the isolated BHET isolated is highly pure.

### FE-SEM Micrographs

FE-SEM micrographs (FIG. 16) of (A) Untreated (fresh) PET (B) PET treated with EG (C) PET treated with DMF revealed that ethylene glycol penetrates into the PET surface by cracking the surface which facilitates the reaction and further helps in achieving the desired selectivity of monomer. Further, DMF converts the PET into porous form which helps to achieve the desired conversion in less reaction time. It was evident from the micrographs that ethylene glycol plays the role of attaining good selectivity and DMF plays the role of achieving the desired conversion.

### TECHNICAL ADVANCEMENTS

The present disclosure described hereinabove has several technical advantages including, but not limited to, the realization of a process for the depolymerization of polyethylene terephthalate (PET), wherein the process:
requires less time period;
is performed without the use of ionic liquids or catalysts;
is carried out at mild process conditions;
is simple, sustainable, and cost effective; and
is eco-friendly as the solvent and co-solvent can be recycled and reused.

Thus, the process for the depolymerization of PET, as disclosed in the present disclosure is a sustainable process to depolymerize the PET waste along with the separation and purification of monomers.

Throughout this specification the word “comprise”, or variations such as “comprises” or “comprising, will be understood to imply the inclusion of a stated element, integer or step,” or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

The numerical values given for various physical parameters, dimensions and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

The economy significance details requirement may be called during the examination. Only after filing of this patent application, the applicant can work publically related to present disclosure product/process/method. The applicant will disclose all the details related to the economic significance contribution after the protection of invention.

1.-21. (canceled)

22. A process for the depolymerization of polyethylene terephthalate (PET) for obtaining monomers, said process comprising the following steps:
a. mixing polyethylene terephthalate (PET) flakes, a solvent and a co-solvent in a predetermined weight ratio to obtain a first mixture;
b. heating said first mixture in an inert atmosphere, at a temperature in the range of 150° C. to 210° C. and at a pressure in the range of 35 mbar to 6 bar under stirring at a first predetermined speed for a first predetermined time period to obtain a second mixture comprising a crude of bis(2-hydroxyethyl) terephthalate (BHET) monomers and oligomers, and a solvent mixture;
c. separating said solvent mixture from said crude to obtain a separated solvent mixture and a separated crude;
d. adding at least one fluid medium to said separated crude in a predetermined ratio and stirring at a second predetermined speed for a second predetermined time period to solubilize said BHET monomers to obtain floccules of said oligomers;
e. separating said floccules of said oligomers to obtain a cake and a third mixture comprising BHET monomers and said fluid medium; and
f. isolating said BHET monomers from said third mixture.

23. The process as claimed in claim 22, wherein said process is a continuous process or a batch process.

24. The process as claimed in claim 22, wherein said predetermined weight ratio of the polyethylene terephthalate (PET) flakes to said solvent to said co-solvent is in the range of 1:5:5 to 1:10:10.

25. The process as claimed in claim 24, wherein said predetermined weight ratio of the polyethylene terephthalate (PET) flakes to said solvent to said co-solvent is 1:7.5:7.5.

26. The process as claimed in claim 22, wherein said solvent is ethylene glycol.

27. The process as claimed in claim 22, wherein said co-solvent is at least one selected from the group consisting of tetrahydrofuran, formamide, N-methylformamide, Dimethylformamide (DMF), Dimethylacetamide (DMAc) Dimethylcarbonate (DMC), dimethyl sulfoxide (DMSO), 1-Methylimidazole and N-Methyl-2-pyrrolidone (NMP).

28. The process as claimed in claim 27, wherein said co-solvent is at least one selected from the group consisting of Dimethylformamide (DMF), Dimethylacetamide (DMAc) and Dimethylcarbonate (DMC).

29. The process as claimed in claim 22, wherein said first predetermined speed is in the range of 500 rpm to 1500 rpm; and said first predetermined time period is in the range of 20 minutes to 120 minutes.

30. The process as claimed in claim 29, wherein said first predetermined speed this 1000 rpm; and said first predetermined time period is 30 minutes.

31. The process as claimed in claim 22, wherein said solvent mixture is separated from said crude by distillation at a temperature in the range of 50° C. to 240° C., and at a pressure in the range of 10 mbar to 1020 mbar.

32. The process as claimed in claim 31, wherein said solvent mixture is separated from said crude by distillation at a temperature in the range of 70° C. to 200° C., and at a pressure of 800 mbar.

33. The process as claimed in claim 22, wherein said fluid medium is at least one selected from the group consisting of water, methanol, ethanol and acetonitrile.

34. The process as claimed in claim 33, wherein said fluid medium is water.

35. The process as claimed in claim 22, wherein a ratio of said fluid medium to said crude is in the range of 5:1 to 25:1, preferably 11:1.

36. The process as claimed in claim 22, wherein said second predetermined speed is in the range of 100 rpm to 700 rpm and said second predetermined time period is in the range of 5 minutes to 30 minutes.

37. The process as claimed in claim 36, wherein said second predetermined speed is 500 rpm and said second predetermined time period is 15 minutes.

38. The process as claimed in claim 22, wherein said isolation of said BHET monomers is done by using at least one method selected from crystallization, drying, evaporation and flash distillation.

39. The process as claimed in claim 38, wherein
said crystallization is performed at a temperature in the range of 0° C. to 10° C.;
said drying by distillation is performed at a temperature in the range of 70° C. to 100° C. at a pressure in the range of 100 mbar to 1020 mbar; and
said evaporation or said flash distillation is performed at a temperature in the range of 50° C. to 110° C. and at a pressure in the range of 35 mbar to 1020 mbar.

40. The process as claimed in claim 22, wherein said BHET monomers are characterized by having:
a purity greater than 98%;
a crystallinity greater than 67%; and
a particle size less than 10 microns.

41. The process as claimed in claim 40, wherein said BHET monomers are characterized by having:
a purity of 98.75%;
a crystallinity in the range of 67.5% to 72.2%; and
a particle size in the range of 0.2 microns to 7.3 microns.
